(19) United States
(12) Patent Application Publication
(10) Pub. No.: US 2023/0068687 A1
(43) Pub. Date: Mar. 2, 2023

(54) PROCESS FOR PREPARING A SOLID FORM OF BASIC AMINO ACID SALTS OF POLYUNSATURATED FATTY ACIDS

(71) Applicant: SILICYCLE INC., Québec (CA)

(72) Inventors: Xiaowei WU, Québec (CA); Christophe MELLON, Québec (CA)

(21) Appl. No.: 17/792,289

(22) PCT Filed: Jan. 29, 2021
(86) PCT No.: PCT/CA2021/050094

§ 371 Date: Jul. 12, 2022

Related U.S. Application Data
(60) Provisional application No. 62/967,657, filed on Jan. 30, 2020.

(51) Int. Cl. A23L 33/12; C11C 1/02; C11C 1/08

(52) U.S. Cl. CPC A23L 33/12; C11C 1/02; C11C 1/08

(57) ABSTRACT

The present disclosure relates to the basic amino acid salts of polyunsaturated fatty acids (PUFAs), and a process for producing same comprising mixing one or more PUFAs in an acid form and a basic amino acid in a mixture of a first organic solvent and water at a temperature of between about above 0° C. to about the boiling point of said first organic solvent; adding a second organic solvent to said mixture of said first organic solvent and water, in an amount effective for precipitating said basic amino acid salts of PUFAs; and evaporating said first and second organic solvents and water to recover said basic amino acid salts of PUFAs.

### FIELD OF THE DISCLOSURE

The present disclosure relates to the basic amino acid salts of polyunsaturated fatty acid (PUFAs), and a process for producing same.

### BACKGROUND

In the 1980s, several publications have revealed that the traditional Greenlandic diet, rich in marine mammals and fish, has substantially lowered mortality from ischaemic heart disease (IHD) in the Inuit population and Danish settlers, albeit to different levels. Such fact is believed to contribute to the effects of polyunsaturated fatty acids (PUFAs) in the traditional marine diet.

Interest in omega-3 has escalated in recent years because of many positive effects on human beings, such as anti-inflammatory and anti-blood clotting actions, lowering triglyceride (TAG) levels, reducing blood pressure, and reducing the risks of diabetes, some cancers, etc.

However, the use of PUFAs as a food additive or food supplement is often limited by stability problems as well as an unpleasant taste and odor.

### SUMMARY OF THE DISCLOSURE

An aspect relates to a process for producing at least one basic amino acid salt of one or more polyunsaturated fatty acids (PUFAs), the process comprising: mixing one or more PUFA in an acid form and a basic amino acid in a mixture of a first organic solvent and water at a temperature of between about above 0° C. to about the boiling point of said first organic solvent; adding a second organic solvent to said mixture of said first organic solvent and water, in an amount effective for precipitating said basic amino acid salts of PUFAs; and evaporating said first and second organic solvents and water to recover said basic amino acid salts of PUFAs.

In an embodiment, the basic amino acid salts of PUFAs are in a solid form.

In a further embodiment, the PUFAs are comprising at least one of omega-3 and omega-6 PUFAs.

In another embodiment, the omega-3 PUFAs are comprising at least one of docosahexaenoic acid (C22:6n-3) (DHA), eicosapentaenoic acid (20:5n-3) (EPA) and alpha-linolenic acid (C18:3n-3) (ALA).

In an embodiment, the omega-3 PUFAs further comprise at least one of eicosatrienoic acid (C20:3(n-3)) (ETE), eicosatetraenoic acid (C20:4(n-3)) (ETA), heneicosapentaenoic acid (C21:5(n-3)) (HPA), docosapentaenoic acid C22:5(n-3) (DPA), tetracosapentaenoic acid (C24:5(n-3)), and tetracosahexaenoic acid (C24:6(n-3)).

In a further embodiment, the omega-6 PUFAs are comprising at least one of linoleic acid (C18:2n-6) and arachidonic acid (C20:4n-6).

In another embodiment, the omega-6 PUFAs further comprise at least one of eicosadienoic acid (C20:2(n-6)), dihomo-gamma-linolenic acid (C20:3(n-6)) (DGLA), docosadienoic acid (C22:2(n-6)), adrenic acid (C22:4(n-6)), docosapentaenoic acid (C22:5(n-6)), tetracosatetraenoic acid C24:4(n-6), and tetracosapentaenoic acid C24:5(n-6)).

In an embodiment, the PUFAs are comprised in a fat and/or oil.

In a further embodiment, the PUFAs are comprising EPA.

In another embodiment, the PUFAs are comprising DHA.

In an embodiment, the PUFAs are comprised in a tuna oil.

In a further embodiment, the PUFAs comprise 50-55% DHA and 20-25% of EPA wt/wt over the total amount of PUFAs.

In another embodiment, the PUFAs comprise 45-60% DHA and 18-27% of EPA wt/wt over the total amount of PUFAs.

In an embodiment, the PUFAs are comprised in seal oil.

In a further embodiment, the PUFAs comprise 5-40% DHA, 5-45% of EPA and 3-10% DPA wt/wt over the total amount of PUFAs.

In another embodiment, the first organic solvent is comprising methanol, ethanol, isopropanol, butanone, acetone and THF or a mixture thereof.

In an embodiment, the mixing of the one or more PUFA and basic amino acid in a mixture of the first organic solvent and the water is performed until a homogenous solution is obtained.

In a further embodiment, the mixing step comprises providing an organic solution comprising the one or more PUFA in an acid form in the first organic solvent, providing an aqueous solution comprising the basic amino acid and water, and mixing the organic solution and the aqueous solution.

In another embodiment, the basic amino acid is L-lysine.

In an embodiment, the basic amino acid is L-arginine.

In an embodiment, the second organic solvent is at least one of ethanol, acetone or a mixture thereof.

### BRIEF DESCRIPTION OF THE FIGURES

FIG. 1. is a graph showing the peroxide value and anisidine value of lysine salts of PUFAs in function of time.

### DETAILED DESCRIPTION

The disclosure relates to a process for producing basic amino acid salts of PUFAs, which leads to the formation of free-flowing powder in one step.

The term “polyunsaturated fatty acid” or “PUFA” as used herein means fatty acid compounds containing two or more ethylenic carbon-carbon double bonds in their carbon backbone. Two major classes of PUFAs are omega-3 and omega-6 PUFAs, characterized by the position of the final double bond in the chemical structure of PUFAs.

Omega-3 PUFAs refer to the position of the final double bond, which in omega-3, the double bond is between the third and fourth carbon atoms from the “omega” or tail end of the molecular chain.

The three most important omega-3 PUFAs are docosahexaenoic acid (DHA), which has 22 carbons and 6 double bonds beginning with the third carbon from the methyl end and is designated as (C22:6n-3), eicosapentaenoic acid (EPA), which is designated as (20:5n-3), and alpha-linolenic acid (ALA), which is designated as (C18:3n-3).

Other omega-3 PUFAs include: Eicosatrienoic acid (ETE) (C20:3(n-3)), Eicosatetraenoic acid (ETA) (C20:4(n-3)), Heneicosapentaenoic acid (HPA) (C21:5(n-3)), Docosapentaenoic acid (Clupanodonic acid) (DPA) C22:5(n-3), Tetracosapentaenoic acid (C24:5(n-3)), and Tetracosahexaenoic acid (Nisinic acid) (C24:6(n-3)).

Omega-6 PUFAs have their terminal double bond in what is referred to as the omega six-position, meaning the last double bond occurs at the sixth carbon from the omega end of the fatty acid molecule.

Among the omega-6 PUFAs, linoleic acid (C18:2n-6) and arachidonic acid (C20:4n-6) are two of the major omega-6s.

Other omega-6 PUFAs include: Eicosadienoic acid (C20:2(n-6)), Dihomo-gamma-linolenic acid (DGLA) (C20:3(n-6)), Docosadienoic acid (C22:2(n-6)), Adrenic acid (C22:4 (n-6)), Docosapentaenoic acid (Osbond acid) (C22:5(n-6)), Tetracosatetraenoic acid C24:4(n-6), and Tetracosapentaenoic acid C24:5(n-6)).

The terms “fat” and/or “oil” used herein refer to any fat and/or oil containing a level of PUFAs suitable for use in the process described herein. The PUFA esters present in the fat or oil are as alkyl esters, triglycerides, diglycerides or monoglycerides or a mixture thereof. In the case of diglycerides or triglycerides, the glycerol unit may optionally bear a phosphorus derivative (hence the fat and/or oil could be or contain phospholipids).

The “first organic solvent” used herein refers to any organic solvent. The first organic solvent also allows for dissolving said one or more PUFA at least at a ratio of about >0-20 (w/w). Examples of such media include methanol, ethanol, isopropanol, butanone, acetone and THF. Preferably the first organic solvent is ethanol, methanol or a mixture thereof.

The “basic amino acid” used herein refers to any amino acid bearing an amine function on its side chain capable to form a salt with a carboxylic acid. Once dissolved in water, it forms a basic aqueous solution.

The “second organic solvent” used herein means the solvents which can cause the precipitation of the basic amino acid salt of PUFAs.

As discussed above, an aspect relates to a process for producing at least one basic amino acid salt of one or more polyunsaturated fatty acids (PUFAs), the process comprising: mixing one or more PUFA in an acid form and a basic amino acid in a mixture of a first organic solvent and water at a temperature of between about above 0° C. to about the boiling point of said first organic solvent; adding a second organic solvent to said mixture of said first organic solvent and water, in an amount effective for precipitating said basic amino acid salts of PUFAs; and evaporating said first and second organic solvents and water to recover said basic amino acid salts of PUFAs.

In one embodiment, the process is conducted at atmospheric pressure.

In one embodiment, the process is conducted without using an inert gas.

In one embodiment, the process is conducted using an inert gas.

In one embodiment, the mixing of said one or more PUFA and basic amino acid in a mixture of a first organic solvent and water is performed until a homogenous solution is obtained.

In one embodiment, the mixing step is comprising providing an organic solution comprising said one or more PUFA in an acid form in said first organic solvent, providing an aqueous solution comprising said basic amino acid and mixing said organic solution and said aqueous solution.

In one embodiment, the mixing step of said one or more PUFA in an acid form and a basic amino acid in a mixture of a first organic solvent and water is preferably conducted below 50° C. or under “atmospheric condition” (i.e. room temperature (e.g. about 20-25° C.) and atmospheric pressure).

In one embodiment, said basic amino acid salts of PUFAs are in solid form such as, for example, a powder. In a further embodiment, the powder is a free flowing powder.

In one embodiment, the process further comprises a step of subjecting the basic amino acid salts of PUFAs to roughing pumper for at least one day.

In one embodiment, the one or more PUFAs are EPAs comprising over 90% wt/wt of omega-3 PUFAs EPA over the total amount of PUFAs.

In one embodiment, the one or more PUFAs are DHAs comprising over 90% wt/wt of omega-3 PUFAs DHA over the total amount of PUFAs.

In one embodiment, the omega-3 PUFAs are from tuna oil comprising 50-55% wt/wt of DHA and 20-25% wt/wt of EPA, alternatively 45-60% wt/wt of DHA and 18-27% wt/wt of EPA, over the total amount of omega-3 PUFAs.

In one embodiment, the omega-3 PUFAs are from seal oil comprising 5-40% wt/wt of DHA, 5-45% of EPA and 3-10% wt/wt of DPA, over the total amount of omega-3 PUFAs.

In one embodiment, the first organic solvent is ethanol.

In one embodiment, the first organic solvent is methanol.

In one embodiment, the first organic solvent is isopropanol.

In one embodiment, the first organic solvent is butanone.

In one embodiment, the first organic solvent is acetone.

In one embodiment, the basic amino acid is L-lysine.

In one embodiment, the basic amino acid is L-arginine.

The weight ratio of the aqueous solution to basic amino acid is dependent on the nature of the basic amino acid. The aqueous component can be used as the least amount to dissolve the amino acid to up to 10 times of the least amount, wherein said dissolution is achieved when no substantial amount of solid basic amino acid is visually present in the aqueous component, the dissolution being conducted at room temperature (i.e. from about 20-25 degrees Celsius). In one embodiment, the amount used is 2 times of the least amount, alternatively 4 times of the least amount, furthermore at 5 times of the least amount.

In one embodiment, the weight ratio of the aqueous solution to L-lysine is between 0.9 to 1.1. In a further embodiment, said weight ratio is 1.

In one embodiment, the second organic solvent is ethanol.

In one embodiment, the second organic solvent is acetone.

Alternatively, the second organic solvent is a mixture of ethanol and acetone.

In one embodiment, the weight ratio of the second organic solvent to said one or more PUFA is 10:1 to 100:1, 10:1 to 70:1, 10:1 to 50:1, and preferably 10:1 to 30:1.

The exact stoichiometry of basic amino acid equivalent to said one or more PUFA in free acid form is difficult to establish with certainty when using fat or oil because of, for example, the indefinite molecular weight of fish oil. In addition, the sources of fish oil differ from one another and may contain different species proportions, such as the variety of proportion of ω-3 composition, ω-6 composition and saturated fatty acids. However, the skilled person can easily estimate the molecular weight in order to carry the invention by making the assumption that the carbon length of fatty acids composition is in the range of C14-C24. Accordingly, as encompassed herein, the fatty acids have an average length of carbon chain of C19. Thus, the molecular weight of 300 g/mol is used herein to estimate the amount of basic amino acids used for the formation of fatty acid salts.

The amount of basic amino acid required to fabricate the fatty acid salt is 1-1.5 mole of basic amino acid for every mole of fatty acid, preferably 1 mole would be sufficient.

The rotor-stator homogenizer is used for the mixing process. Typically, the homogenizer speed is from 50 rpm to 1000 rpm, and preferably from 100-200 rpm.

The final product in powder form is isolated by evaporating said first and second organic solvents and water from the reaction mixture to recover said basic amino acid salts of PUFAs. Preferably, said evaporation step is carried under reduced pressure between about 0° C.-70° C. depending on the properties of the equipment used. The evaporation step described herein does not contemplate the use of methods such as spray drying, where the spraying nozzle often requires an inlet air temperature above 100° C. The evaporation step also does not contemplate including freeze-drying methods. The evaporation step requires said first/second organic solvents and water to be in a liquid form, thereby excluding the freeze-drying methods that require the solvents to be frozen. The oxidative status of the obtained final product is quantified by peroxide value (PV), anisidine value (AV) and Totox value. PV is a measure of the level of the primary oxidation products (lipid hydroperoxides) in the product, which is specified in milliequivalents O₂ per kg of sample, while the AV is an unspecific measure of saturated and unsaturated carbonyl compounds. Totox is calculated by the equation Totox=2*PV+AV.

The comparison of oxidative status of basic amino acid salts of PUFAs, starting material in ester form and fish oil in free acid form are assessed by measuring the PV and AV, then all the samples are subjected to the same oxidizing condition over a certain period of time, followed by the measuring PV and AV of the samples. The oxidizing conditions are selected from one of: 1) storage in closed containers at atmospheric condition, opened once daily for the first month, twice weekly for the second month, once weekly for the third month and twice a month up to 6 months; 2) storage in loosely closed containers exposed to air at 45° C. for 1 month.

In one embodiment, the basic amino acid salts of PUFAs are synthesized following the general procedure where the one or more PUFAs comprise eicosapentaenoic acid (EPA) at a concentration of >90% wt/wt over the total amount of PUFAs. The molar percent range of EPA to the basic amino acid is 30%-50%/70%-50%, 40-50%/60-50% and 45-50%/55-50% respectively, and preferentially the molar percent ratio is 50%/50%. The solvents are removed at reduced pressure 0-70 mm Hg at 0° C.-70° C., preferentially at 30 mm Hg at 40° C., followed by the roughing pump for at least a day.

In one embodiment, the basic amino acid salts of PUFAs are synthesized following the general procedure where the one or more PUFAs comprise docosahexaenoic acid (DHA) at a concentration of >90% wt/wt over the total amount of PUFAs. The molar percent range of DHA to the basic amino acid is 30%-50%/70%-50%, 40-50%/60-50% and 45-50%/55-50% respectively, and preferentially the molar percent ratio is 50%/50%. The solvents are removed at reduced pressure 0-70 mm Hg at 0° C.-70° C., preferentially at 30 mmHg at 40° C., followed by the roughing pump for at least a day.

In one embodiment, the basic amino acid salts of PUFAs are synthesized following the general procedure where the one or more PUFAs are tuna oil as free acid containing 50-56% DHA and 20-25% of EPA wt/wt over the total amount of PUFas. The molar percent range of tuna oil to the basic amino acid is 30%-50%/70%-50%, 40-50%/60-50% and 45-50%/55-50% respectively, and preferentially the molar percent ratio is 50%/50%. The solvents are removed at reduced pressure 0-70 mm Hg at 0° C.-70° C., preferentially at 30 mm Hg at 40° C., followed by the roughing pump for at least a day.

In one embodiment, the basic amino acid salts of PUFAs are synthesized following the general procedure where the one or more PUFAs are seal oil as free acid with 5-40% DHA, 5-45% of EPA and 3-10% DPA wt/wt over the total mount of PUFAs. The molar percent range of seal oil as free acid to the basic amino acid is 30%-50%/70%-50%, 40-50%/60-50% and 45-50%/55-50% respectively, and preferentially the molar percent ratio is 50%/50%. The solvents are removed at reduced pressure 0-70 mmHg at 0° C.-70° C., preferentially at 30 mmHg at 40° C., followed by the roughing pump for at least a day.

### Sample Characterization

All the reagents and solvents used herein are commercially available without further purification. GC-MS used here is Agilent 5977B/7890B, and the column is Agilent HP-5 ms-UI.

Food Lab Analyzer: Among several techniques known in the art for determining the oxidative levels of a sample, CDR FoodLab® Junior analyzer is used herein for determining PV and AV. The procedures are described as below.

The solid product 0.5 g was dissolved in 2 mL of MeOH and HCl solution with the ratio of 1:10 (v/v). The mixture was stirred for 5 minutes, followed by the addition of 5 mL of water. The mixture was extracted with 3 mL of Hexane containing 100 ppm butylated hydroxytoluene (BHT). The organic layer was dried over MgSO₄, filtrated and evaporated under reduced pressure at the temperature of 0-70° C. to get the fish oil in free acid form, which was evaluated with the CDR FoodLab® Junior analyzer to get anisidine and peroxide values using the Food Lab analyzer.

Gas chromatography-mass spectrometry (GC-MS): The PUFAs concentrates of the final product were determined by gas chromatography-mass spectrometry (GC-MS).

Esterification of PUFAs: Around 25 mg of FFA or FFA salts (free fatty acid) was charged in a sealed tube, and 2 mL of a solution of 2% H₂SO₄ is added to generate a homogenous solution, which was then heated (without any agitation) at 80° C. for 30 minutes, followed by the addition of 2 mL of saturated NaHCO₃ aqueous solution after the solution was cooled down to room temperature. The FFA in ester form was extracted with 8-10 mL of 100 ppm BHT hexanes once. Subsequently, the organic layer was dried over MgSO₄ analysed by GC-MS.

Water Quantification in spheroidal organosiloxane sub-micron/nanoparticles (Karl Fisher): The water percentage was estimated by using titrator Compact V20s from Mettler Toledo.

Flowability: The flowability was determined by using the US Pharmacopoeia method from the General Information number 1174 (Powder Flow 801).

### EXAMPLES

### Example 1: The Preparation of Tuna Oil in Free Acid from PUFAs Ethyl Ester of Tuna Oil

A 2 L of 3-neck round bottle glassware was charged with 200 mL of ethanol, and followed by the addition of 64 g of 50% of NaOH aqueous solution. Subsequently, PUFAs ethyl ester of tuna oil with an AV of 6.8 A/g and PV of 13 meqO₂/kg was dropped to the mixture under nitrogen and stirred at the speed of 150 rpm with the overhead stirrer. The resulting solution was stirred for 1.5 hour at room temperature. After cooling down to the room temperature, 600 mL of H₂O and 60 mL H₃PO₄ (85%) were added, which was stirred for 10 minutes. The organic phase was then extracted with 60 mL of hexane once and was dried over MgSO₄. After removing the organic solvent, 170 g of tuna oil in free acid form with EPA of 23.7% and DHA of 55.6% was generated as oil with a PV of 0.96 meqO₂/kg and an AV of 1.5 A/g.

### Example 2: The Preparation of L-Lysine Salt of Eicosapentaenoic Acid (EPA-Lys)

A 250 mL round bottle flask was first charged with 3 g of EPA, exhibiting a PV of >50 meqO₂/kg and an AV of >100 A/g, and 3 g of ethanol, followed by the addition of 3 g of 50% of L-lysine aqueous solution. The molar ratio of EPA to L-lysine was 1:1. The mixture was stirred at atmospheric condition for 5 minutes to obtain a homogenous solution. 30 mL of ethanol was added to precipitate the L-lysine amino acid salt. Subsequently, the solvents were evaporated under reduced pressure 30 mm Hg at 40° C. to achieve the final product as brown powder, which was further subject to roughing pumper for 1 day to generate EPA-Lys with a PV of 6.0 meqO₂/kg and an AV of 9.5 A/g. About 4.5 g of EPA-Lys was stored in a 20 ml of clear vial with a diameter of 27 mm, which was placed with closed lids on the bench at room temperature for the stability examination for 90 days. The results are summarized in table 1.

### Example 3: The Preparation of L-Lysine Salt of Docosahexaenoic Acid (DHA-Lys)

A 250 mL round bottle flask was first charged with 3 g of DHA, a PV of >50 meqO₂/kg and an AV of >100 A/g, 3 g of ethanol, followed by the addition of 3 g of 50% of L-lysine aqueous solution. The molar ratio of DHA to L-lysine was 1:1. The mixture was stirred at atmospheric condition for 5 minutes to obtain a homogenous solution. 30 mL of ethanol was added to precipitate the L-lysine amino acid salt. Subsequently, the solvents were evaporated under reduced pressure 30 mm Hg at 40° C. to achieve the final product as brown powder, which was further subject to roughing pumper for 1 day to generate DHA-Lys with a PV of 16.0 meqO₂/kg and an AV of 29.3 A/g. About 4.5 g of EPA-Lys was stored in a 20 ml of clear vial with a diameter of 27 mm, which was placed with closed lids on the bench at room temperature for the stability examination for 90 days. The results are summarized in table 1.

TABLE 1
Assessment of stability of L-lysine salt of DHA (DHA-
Lys), EPA (EPA-Lys) and Seal oil (Seal oil-Lys)

| | | PV/meq | | |
|---|---|---|---|---|
| Sample | Time/d | O₂/Kg | AV/A/g | TOTOX |
| DHA-OH | Starting | >50 | >100 | — |
| | material | | | |
| EPA-OH | Starting | >50 | >100 | — |
| | material | | | |
| DHA-Lys | 1 | 16.0 | 29.3 | 61.3 |
| | 61 | 3.9 | 4.0 | 11.8 |
| EPA-Lys | 1 | 6.0 | 9.5 | 21.5 |
| | 61 | 1.9 | <0.5 | 3.8 |
| Seal oil-Lys | 1 | 1.5 | <0.5 | 3.0 |
| | 61 | 0.7 | <0.5 | 1.4 |

### Example 4: The Preparation of L-Lysine Salt of Tuna Oil in Free Acid Form (EPA of 23.7% and DHA of 55.6%) (PUFAs-Lys)

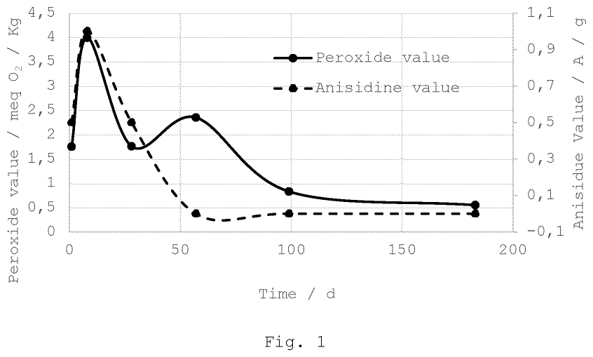

A 1 L round bottle flask was first charged with 10 g of tuna oil in free acid form prepared from example 1, and 10 g of ethanol, followed by the addition of 10.6 g of 50% of L-lysine aqueous solution. The molar ratio of tuna oil in free acid form to L-lysine was 1:1. The mixture was stirred at atmospheric condition for 5 minutes to obtain a homogenous solution. 300 mL of ethanol was added to precipitate the L-lysine amino acid salt. Subsequently, the solvents were evaporated under reduced pressure 30 mm Hg at 40° C. to achieve the final product as beige powder, which was further subject to roughing pumper for 1 day to generate PUFAs-Lys with a PV of 1.8 meqO₂/Kg and an AV of 0.5 A/g. About 15 g of EPA-Lys was stored in a 120 ml of clear bottle with a diameter of 48 mm which was placed with closed lids on the bench at room temperature for the stability examination for 183 days. The results are presented in FIG. 1.

### Example 5: The Preparation of L-Lysine Salt of Seal Oil in Free Acid Form (EPA of 20.2%, DHA of 25.3% and DPA of 7.7%) (Seal Oil-Lys)

A 250 mL round bottle flask was first charged with 3 g of seal oil in free acid form, exhibiting a PV of 10.6 meqO₂/kg and an AV of 5.7 A/g, and 3 g of ethanol, followed by the addition of 6 g of 50% of L-lysine aqueous solution. The molar ratio of seal oil in free acid form to L-lysine was 1:1. The mixture was stirred at atmospheric condition for 5 minutes to obtain a homogenous solution. 35 mL of ethanol was added to precipitate the L-lysine amino acid salt. Subsequently, the solvents were evaporated under reduced pressure 30 mm Hg at 40° C. to achieve the final product as beige powder, which was further subject to roughing pumper for 1 day to generate the amino acid salts of PUFAs with a PV of 1.5 meqO₂/kg and an AV of <0.5 A/g. The stability of Seal oil-Lys is as in table 1.

### Example 6: The Preparation of L-Arginine Salt of Tuna Oil in Free Acid Form (EPA of 23.7% and DHA of 55.6%) (PUFAs-Arg)

A 250 mL round bottle flask was first charged with 3 g of tuna oil in free acid form prepared from example 1, and 30 g of ethanol, followed by the addition of 2.70 g of 63% of L-arginine aqueous solution. The molar ratio of tuna oil in free acid form to L-arginine was 1:1. The mixture was stirred at atmospheric condition until a homogenous solution was obtained. 300 mL of acetone was added to precipitate the L-arginine amino acid salt. Subsequently, the solvents were filtered off to produce the final product as pinkish powder, which was further subject to roughing pumper for 1 day to generate PUFAs-Lys with a PV of 2.6 meqO₂/Kg, and an AV of 0.7 A/g.

### Example 7: Assessment of Stability of Amino Acid Salt of PUFAs (PUFAs-Lys) and PUFAs in the Form of Ester (PUFAs-OEt) and Free Acid (PUFAs-OH)

About 5 g of the liquid tuna oil ethyl ester (PUFAs-OEt) used in example 1, the tuna oil in free acid form (PUFAs) obtained in example 1 and its corresponding L-lysine salt (PUFAs-Lys), were added into 20 ml of clear vial with a diameter of 27 mm. The three vials were placed with closed lids on the bench at room temperature. The analyses were performed after 30 days. Based on the results, stability of L-lysine salt of omega-3 was further assessed by storing the vial with opened lids in an oven with an opened ventilation at 45° C. for another 30 days. The obtained results are summarized in table 2.

TABLE 2
Assessment of stability of L-lysine salt of
PUFAs (PUFAs-Lys) and PUFAs in the form of
ester (PUFAs-OEt) and free acid (PUFAs-OH)

| | | PV/meq | | |
|---|---|---|---|---|
| Sample | Time/d | O₂/Kg | AV/A/g | TOTOX |
| PUFA-OEt | 1 | 1.2 | <0.5 | 2.4 |
| | 30 | >50 | 28.1 | — |
| PUFA-OA | 1 | 31.9 | 18.7 | 82.5 |
| | 30 | >50 | 24.2 | — |
| PUFA-Lys | 1 | 0.44 | <0.5 | 0.88 |
| | 30 | 0.45 | <0.5 | 0.90 |
| | 63 * | 0.53 | <0.5 | 1.06 |

Note:
* The sample is placed at 45° C. with opened lid during the period of 30-63 days.

While the present disclosure has been described in connection with specific embodiments thereof, it will be understood that it is capable of further modifications and this application is intended to cover any variations, uses, or adaptations including such departures from the present disclosure as come within known or customary practice within the art and as may be applied to the essential features hereinbefore set forth, and as follows in the scope of the appended claims.

1. A process for producing at least one basic amino acid salt of one or more polyunsaturated fatty acids (PUFAs), the process comprising:
mixing one or more PUFAs in an acid form and a basic amino acid in a mixture of a first organic solvent and water at a temperature of above 0° C. to about the boiling point of said first organic solvent;
adding a second organic solvent to said mixture of said first organic solvent and water, in an amount effective for precipitating said basic amino acid salts of PUFAs; and
evaporating said first and second organic solvents and water to recover said basic amino acid salts of PUFAs.

2. The process of claim 1, wherein said basic amino acid salts of PUFAs are in a solid form.

3. The process of claim 1, wherein said PUFAs are comprising at least one of omega-3 and omega-6 PUFAs.

4. The process of claim 3, wherein said omega-3 PUFAs are comprising at least one of docosahexaenoic acid (C22:6n-3) (DHA), eicosapentaenoic acid (20:5n-3) (EPA) and alpha-linolenic acid (C18:3n-3) (ALA).

5. The process of claim 3, wherein said omega-3 PUFAs further comprise at least one of eicosatrienoic acid (C20:3(n-3)) (ETE), eicosatetraenoic acid (C20:4(n-3)) (ETA), heneicosapentaenoic acid (C21:5(n-3)) (HPA), docosapentaenoic acid C22:5(n-3) (DPA), tetracosapentaenoic acid (C24:5(n-3)), and tetracosahexaenoic acid (C24:6(n-3)).

6. The process of claim 3, wherein said omega-6 PUFAs are comprising at least one of linoleic acid (C18:2n-6) and arachidonic acid (C20:4n-6).

7. The process of claim 3, wherein said omega-6 PUFAs further comprise at least one of eicosadienoic acid (C20:2(n-6)), dihomo-gamma-linolenic acid (C20:3(n-6)) (DGLA), docosadienoic acid (C22:2(n-6)), adrenic acid (C22:4(n-6)), docosapentaenoic acid (C22:5(n-6)), tetracosatetraenoic acid C24:4(n-6), and tetracosapentaenoic acid C24:5(n-6)).

8. The process of claim 1, wherein said PUFAs are comprised in a fat and/or oil.

9. The process of claim 1, wherein said PUFAs are comprising EPA.

10. The process of claim 1, wherein said PUFAs are comprising DHA.

11. The process of claim 1, wherein said PUFAs are comprised in a tuna oil or seal oil.

12. The process of claim 10, wherein said PUFAs comprise 50-55% DHA and 20-25% of EPA wt/wt over the total amount of PUFAs.

13. The process of claim 10, wherein said PUFAs comprise 45-60% DHA and 18-27% of EPA wt/wt over the total amount of PUFAs.

14. (canceled)

15. The process of claim 14, wherein said PUFAs comprise 5-40% DHA, 5-45% of EPA and 3-10% DPA wt/wt over the total amount of PUFAs.

16. The process of claim 1, wherein said first organic solvent is comprising methanol, ethanol, isopropanol, butanone, acetone and THF or a mixture thereof.

17. The process of claim 1, wherein said mixing of said one or more PUFA and basic amino acid in a mixture of said first organic solvent and said water is performed until a homogenous solution is obtained.

18. The process of claim 1, wherein said mixing step comprises providing an organic solution comprising said one or more PUFA in an acid form in said first organic solvent, providing an aqueous solution comprising said basic amino acid and water, and mixing said organic solution and said aqueous solution.

19. The process of claim 1, wherein said basic amino acid is L-lysine.

20. The process of claim 1, wherein said basic amino acid is L-arginine.

21. The process of claim 1, wherein said second organic solvent is at least one of ethanol, acetone or a mixture thereof.
